(19) United States
(12) Patent Application Publication
(10) Pub. No.: US 2006/0110291 A1
(43) Pub. Date: May 25, 2006

(54) Electronic sensing circuit

(76) Inventor: Qiong Wang, Shanghai (CN)

Correspondence Address: Wang Qiong, 235 Chung - Ho, Box 8-24, Taipei, TW

(21) Appl. No.: 11/041,149

(22) Filed: Jan. 24, 2005

(30) Foreign Application Priority Data
Nov. 25, 2004 (TW) 093218952

(51) Int. Cl. G01N 33/00

(52) U.S. Cl. 422/68.1

(57) ABSTRACT

An electronic sensing circuit serves for sensing a disposable sensing element. The disposable sensing element has a diode. The biological sensing circuit comprises a power control unit as a power control of the circuit; a slot for locating the disposable sensing element; a reaction section; a reference voltage unit provides different reference voltages based on the kind of the disposable sensing element to the disposable sensing element; a temperature sensing unit for measuring the temperature of an object to be sensed; a main control unit for, controlling the operation of the circuit; a display unit for displaying the measuring result; an input unit for inputting instructions; and a memory unit for storage the measuring data. The voltage has different forms determined by the main control unit or other device connected thereto. Thereby, the present invention can be used with different disposable, sensing elements with a higher precision.

### FIELD OF THE INVENTION

The present invention relates to sensors, and in particular to an electronic sensing circuit which provides different reference voltages to the circuit based on the kind of a disposable sensing element. The voltage may have different forms determined by the main control unit or other device connected to the main control unit. Thereby, the present invention can be used with different disposable sensing elements with a higher precision.

### BACKGROUND OF THE INVENTION

Currently, biological sensing circuits are used to detect glucose, cholesterol, and hemoglobin in the blood. The biological sensing circuit serves to detect the disposable sensing elements. When the disposable sensing element is inserted into the biological sensing circuit, blood is dropped upon a reaction section of the disposable sensing element so that the diode of the reaction diode is conductive. Thereby the diode in the reaction section is conductive. The biological sensing circuit will measure and analyze the input signals so as to analyze the density of the glucose, cholesterol, and hemoglobin.

However the development of the hardware of the biological sensing circuit can not mach the development of the biological sensing circuit so that the user must update the biological sensing circuit. Thereby the biological sensing circuit has specific disposable sensing elements. Thus, the biological sensing circuit must update with the disposable sensing element. This is not cost-effective.

In another prior art, the biological sensing circuit applies a discontinuous rectangular wave to the diode of a disposable sensing element. Since the measured current is only one point, the precision is low.

### SUMMARY OF THE INVENTION

Accordingly, the primary object of the present invention provides an electronic sensing circuit, which provides different reference voltages to the circuit based on the kind of a disposable sensing element. The voltage may have different forms, such as one pulse, continuous pulse, pulses of different frequencies, continuous rectangular waves, which can be determined by the main control unit or other device connected to the main control unit. Thereby, the present invention can be used with different disposable sensing elements with a higher precision than the prior art.

To achieve above object, the present invention provides an electronic sensing circuit for sensing a disposable sensing element; the disposable sensing element having a diode. The biological sensing circuit comprises a power control unit as a power control of the circuit; a slot for locating a disposable sensing element; a reaction section; a reference voltage unit provides different reference voltages based on the kind of the disposable sensing element to the disposable sensing element; a temperature sensing unit for measuring the temperature of an object to be sensed; a main control unit for controlling the operation of the circuit; a display unit for displaying the measuring result; an input unit for inputting instructions; a memory unit for storage the measuring data.

The various objects and advantages of the present invention will be more readily understood from the following detailed description when read in conjunction with the appended drawing.

### BRIEF DESCRIPTION OF THE DRAWING

FIG. 1 is a circuit block diagram of the biological sensing circuit of the present invention.

FIG. 2 is a schematic view showing one application of the present invention, wherein a continuous rectangular wave is provided.

FIG. 3 is a schematic view of the present invention, where the measured current is displayed.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

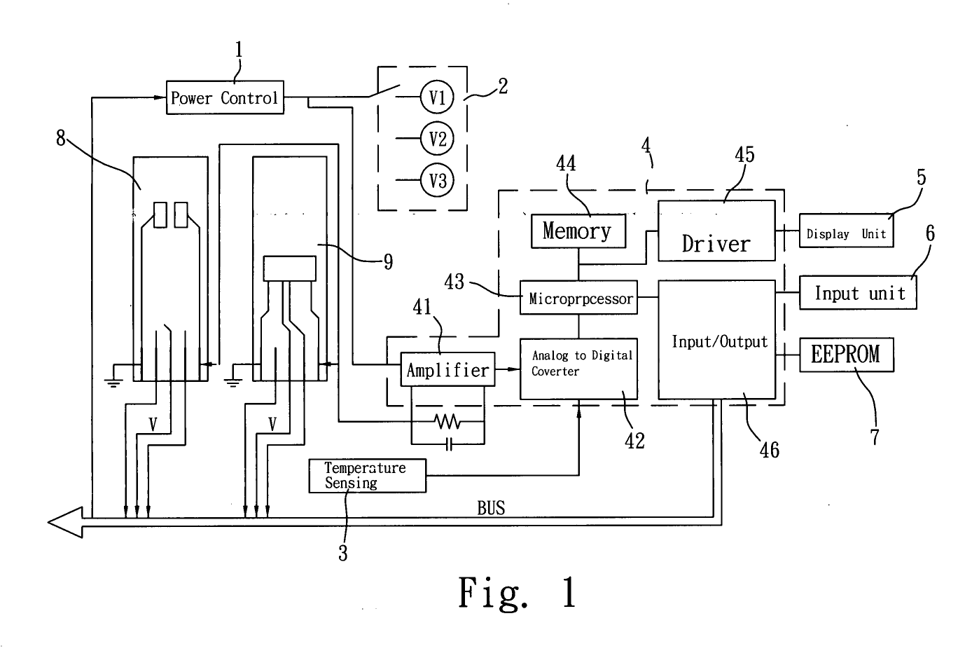

Referring to FIG. 1, a schematic view about the biological sensing circuit of the present invention is illustrated. The present invention has the following elements.

A power control unit 1 serves as a power control of the circuit.

A reference voltage unit 2 provides different reference voltages to the circuit based on the kind of a disposable sensing element. The voltage may have different forms, such as one pulse, continuous pulses, pluses of different frequencies, continuous rectangular waves, which can be determined by the main control unit 4 or other device connected to the main control unit 4. Thereby, the present invention can be used with different disposable sensing elements.

A temperature sensing unit 3 serves for measuring the temperature of an object to be sensed.

A main control unit 4 serves for controlling the operation of the circuit.

A display unit 5 is a display for displaying the measuring result.

An input unit 6 is formed by a plurality of keys for inputting instructions.

A memory unit 7 is a writible memory for storage the measuring data.

In the present invention, the main control unit 4 further comprises the following elements.

An amplifier 41 is connected to the power control unit 1.

An analog to digital converter 42 connected to the amplifier 41 and the temperature sensing unit 3.

An microprocessor 43 is connected to the analog to digital converter 42.

A memory 44 is connected to the microprocessor 43.

A driver 45 is connected to the microprocessor 43 and the display unit 5.

An input/output port 46 is connected to the microprocessor 43, the input unit 6 and the memory unit 7.

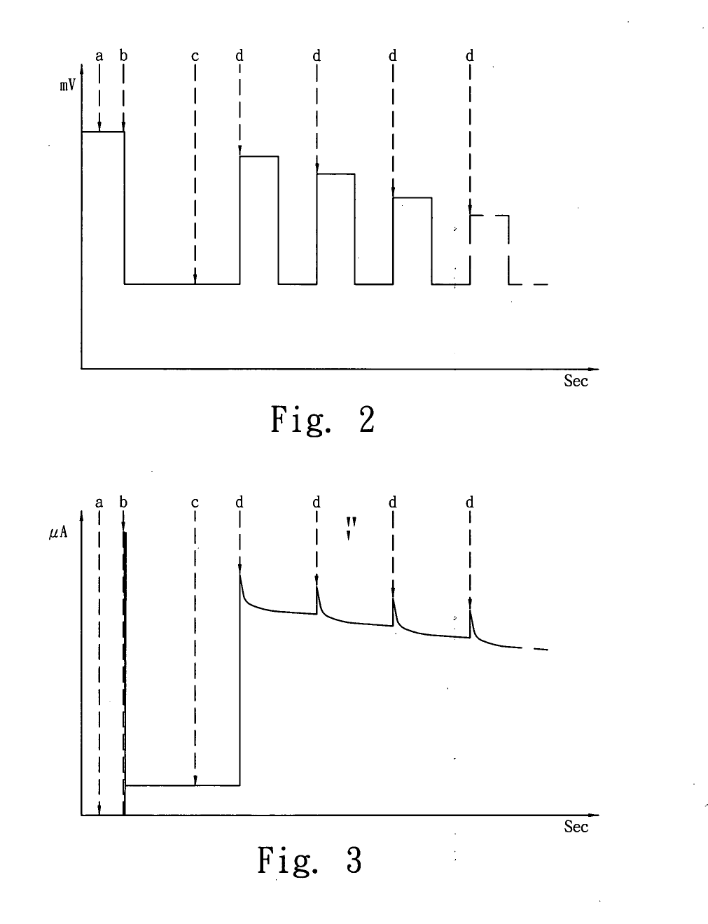

Referring to FIGS. 1, 2, and 3, in the present invention, when a disposable sensing element is inserted into a slot, of the biological sensing circuit, the biological sensing circuit is actuated. Then, the power control unit 1 will actuate the reference voltage unit 2 to provide a continuous rectangular wave to a diode of a disposable sensing element. If no object to be measured (such as blood, urine) is placed on the disposable sensing element, the diode of the disposable sensing element is opened without any current. The disposable sensing element has a reaction section (point A in FIGS. 2 and 3) for receiving the object to be measured.

When the object to be measured is placed to the reaction section, the diode will conduct. If the current flowing through the diode larger than a preset valve, the voltage applied to the diode will be reduced to a set value. Then current will reduce (point B in FIGS. 2 and 3).

Then the object to be measured is reacted with the reagent in the reaction action. The reaction time is determined by the memory unit 7 or other setting element connected to the memory unit 7 (point C in FIGS. 2 and 3).

When the reaction time is over, a measuring operation is performed. The voltage applied and test times are different from objects to objects. For example, if glucose is tested, 2-5 time tests are performed. The voltage is reduced from 400 mV with a step of 5.0 mV (point D in FIGS. 2 and 3). For example, if the density of glucose in blood is tested, a point current is read per 0.5 second. If the total reaction time is 5 second, there are nine sets (data measured at 0.5 second, 1.0 second, 1.5 second, 2.0 second, 2.5 second, 3.0 second, 3.5 second, 4.0 second, 4.5 second, and 5.0 second) current values. An optimum value with a preferred precision and accuracy is displayed on the display unit 5. Thee preferred precision and accuracy are set in the main control unit 4 or other device connected to the main control unit 4.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An electronic sensing circuit for sensing a disposable sensing element; the disposable sensing element having a reaction section and a diode; an object to be measured being placed on the reaction section; the biological sensing circuit comprising:
a power control unit as a power control of the circuit;
a slot for locating the disposable sensing element;
a reference voltage unit provides different reference voltages based on the kind of the disposable sensing element to the disposable sensing element;
a temperature sensing unit for measuring the temperature of an object to be sensed;
a main control unit for controlling the operation of the circuit;
a display unit for displaying the measuring result;
an input unit for inputting instructions; and
a memory unit for storage the measuring data;
wherein when a disposable sensing element is inserted into a slot of the biological sensing circuit, the biological sensing, circuit is actuated; then, power control unit will actuate the reference voltage unit to provide a voltage wave to the diode of the disposable sensing element; if no object to be measured is placed on the disposable sensing element, the diode of the disposable sensing element is opened without any current;
when the object to be measured is placed on the reaction section, the diode will conduct; if the current flowing through the diode larger than a preset valve, the voltage applied to the diode will be reduced to a set value; then current will reduce.

2. The electronic sensing circuit as claimed in claim 1, wherein a reference voltage unit provides different reference voltages to the circuit based on the kind of a disposable sensing element which is determined by the main control unit or other device connected to the main control unit.

3. The electronic sensing circuit as claimed in claim 2, wherein the voltage provided by the reference voltage unit is one of a single pulse, continuous pulses, pulses of different frequencies, continuous rectangular waves.

4. The electronic sensing circuit as claimed in claim 1, wherein the main control unit further comprises:
an amplifier connected to the power control unit;
an analog to digital converter connected to the amplifier and the temperature sensing unit;
a microprocessor connected to the analog to digital converter;
a memory connected to the microprocessor;
a driver connected to the microprocessor and the display unit; and
an input/output port connected to the microprocessor, the input unit and the memory unit.

5. The electronic sensing circuit as claimed in claim 1, wherein the display unit is a display for displaying the measuring result.

6. The electronic sensing circuit as claimed in claim 1, wherein the input unit is formed by a plurality of keys for inputting instructions.

7. The electronic sensing circuit as claimed in claim 1, wherein the memory unit is a writible memory.
